(19) United States
(12) Patent Application Publication
(10) Pub. No.: US 2011/0073810 A1
(43) Pub. Date: Mar. 31, 2011

(54) PROCESS FOR PREPARING ISOTOPICALLY LABELED VITAMINS SUITABLE FOR USE AS ANALYTICAL REFERENCE STANDARDS

(76) Inventors: Scott White Landvatter, King of Prussia, PA (US); Edith Foris Landvatter, King of Prussia, PA (US); Richard Steven Tyburski, Medford, NJ (US)

(21) Appl. No.: 12/837,710

(22) Filed: Jul. 16, 2010

Related U.S. Application Data
(60) Provisional application No. 61/246,262, filed on Sep. 28, 2009.

(51) Int. Cl. G01N 31/00; C07C 401/00

(52) U.S. Cl. 252/408.1; 552/653; 204/158.14

(57) ABSTRACT

The present invention is directed to a method for the synthesis of deuterium labeled Vitamin D and related compounds with a high level of deuterium incorporation, which are particularly suitable for use as standards for mass spectroscopy.

### CROSS REFERENCE TO RELATED APPLICATIONS

This application claims priority to U.S. Provisional Application No. 61/246,262, filed Sep. 9, 2009, which is incorporated by reference in its entirety.

### FIELD OF THE INVENTION

The invention relates to a novel method of producing deuterium labeled compounds including Vitamin D and related analogues and the composition of matter of the labeled compounds. The invention also relates to the use of the labeled compounds for reference standards for mass spectroscopic analysis.

### BACKGROUND OF THE INVENTION

Cholecalciferol (Vitamin D3) and ergocalciferol (Vitamin D2) are important vitamins that mediate calcium absorption. Each vitamin serves as a precursor to the 25-hydroxy metabolite and ultimately to the physiologically active 1,25-dihydroxy metabolites. Other biologically important vitamin metabolites include their respective 24,25-dihydroxy metabolites.

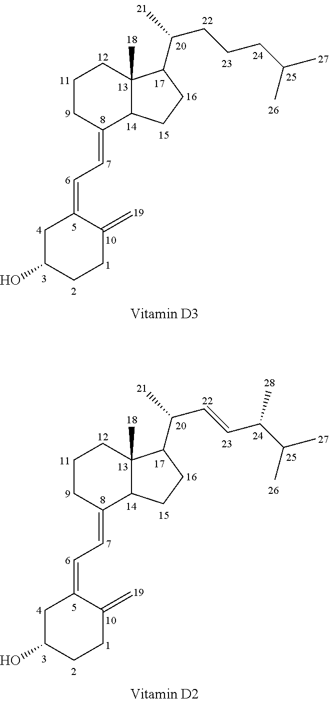

Detection and quantification of these and other vitamin D-related metabolites are important to assess various conditions related to vitamin deficiencies. Current methodology, in particular those methods relying upon radioimmunoassay (RIA), are not entirely unambiguous and can fail to discriminate between parent vitamins and their metabolites.

New analytical detection and quantification techniques involving mass spectroscopy are capable of discriminating between all vitamin D related compounds. Accurate quantification requires that a suitable stable isotope labeled reference standard be available such that the reference standard has a molecular weight enhancement of at least +2.97 amu and contains less than 0.1% unlabeled parent compound.

A general method for labeling vitamin D related compounds was first reported by Reischl and Zbiral in Helvetica Chimica Acta 62(6) 1763 (1979) and involves the activation of vitamin D compounds by cycloaddition of sulfur dioxide to yield a cyclic sulfone. Hydrogen atoms alpha to the sulfone are rendered acidic and are susceptible to exchange with deuterium under basic conditions and a suitable source of deuterium. Various bases and solvents have been described in the literature including sodium bicarbonate in dimethylformamide (Yamada, et. Al., Tetrahedron Letters, 22(32) 3085 (1981)), potassium t-butoxide in DMF (Ray, et. al., Steroids 57 142 (1992)), and sodium isopropoxide in isopropanol or sodium methoxide in methanol (Iwasaki, et. al., Steroids 64 396 (1999)). Elimination of the sulfur dioxide followed by isomerization of the thus obtained triene yields a vitamin D or vitamin D analog labeled with up to three deuterium atoms at carbons 6 and 19.

The primary drawback of all these methods is typified by the reported deuteration of 24,25-dihydroxyvitamin D3 by Yamada, et. al. (Steroids 54(2) 145 (1989)) where total deuterium incorporation is only 2. 6 D/molecule which is far too little to be of any real value for use as a labeled standard.

### SUMMARY OF THE INVENTION

One embodiment of the invention involves a process for preparing the vitamin D and vitamin D-related deuterium labeled compounds shown below suitable for use as analytical reference standards for assays involving mass spectroscopy.

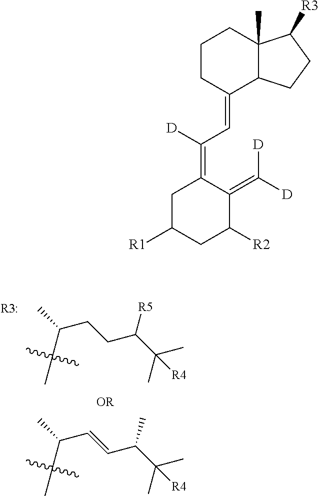

where R1=H or OH, R2=H or OH and R3 can be any saturated or unsaturated side chain including those typical of a steroid or vitamin: where R4=H or OH and R5=H or OH.

Another embodiment of the invention involves the reaction of the 7Z isomer of a vitamin D or vitamin D analog with sulfur dioxide to facilitate the deuterium labeling of said compounds.

Still another embodiment of the invention involves deuterium labeled compounds including Vitamin D and related analogues that can be produces by the methods described above and the use of the labeled compounds for reference standards for mass spectroscopic analysis.

Other objects and advantages of the invention will be apparent to those skilled in the art from the following detailed description of the invention.

### DETAILED DESCRIPTION OF THE INVENTION

This invention relates to a novel process for the deuterium labeling of vitamin D and vitamin D analogs and novel compounds produced by the process. The final deuterated compounds so produced have been found to have greater than 2.97 deuterium atoms per molecule and to contain less than 0.1% unlabeled vitamin, and thus are suitable to serve as an analytical reference standards for mass spectroscopy. This invention therefore affords significant advantages over any existing methodology.

This invention is specifically suitable for the production of deuterium labeled vitamin D3, vitamin D2, 25-hydroxyvitamin D3, 25-hydroxyvitamin D2, 1,25-dihydroxyvitamin D3, 1,25-dihydroxyvitamin D2, 24,25-dihydroxyvitamin D3 and 24,25-dihydroxyvitamin D2. Similarly, all such vitamin and steroid related analogs having a characteristic 5, 7, 10(19) triene system can be labeled with deuterium in accordance with the present invention, to the levels described herein.

In an example of the practice of the invention, the first steps of the process requires activation of the appropriate triene system with sulfur dioxide, exchange of deuterium into the activated positions and subsequent extrusion of the sulfur dioxide. Purification of the partially labeled intermediate thus obtained is followed by a second activation step with sulfur dioxide, a second exchange labeling procedure, and further extrusion of the sulfur dioxide.

The second sulfur dioxide addition may be done on either the 7E or 7Z isomer of the partially labeled vitamin, preferably on the 7Z isomer. This is the first report of SO₂ activation on such a 7Z isomer and represents a novel use of this activation method on a vitamin or vitamin related compound bearing a double bond at position 7.

Photoisomerization of the labeled vitamin followed by chromatographic purification gives the deuterated vitamin with deuterium incorporation of at least 2.97 D/molecule and less than 0.1% unlabeled vitamin remaining, making said product suitable as an analytical reference standard for mass spectroscopy.

The procedure described above in the practice of the process of the present invention will produce the following general structure with deuterium incorporated at the positions shown:

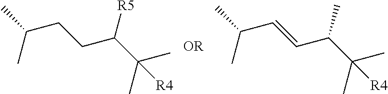

where R1=H or OH, R2=H or OH and R3 can be any saturated or unsaturated side chain including those typical of a steroid or vitamin: where R4=H or OH and R5=H or OH.

A preferred embodiment of the invention is shown in Scheme 1, below, where R1, R2, and R3 are as shown above.

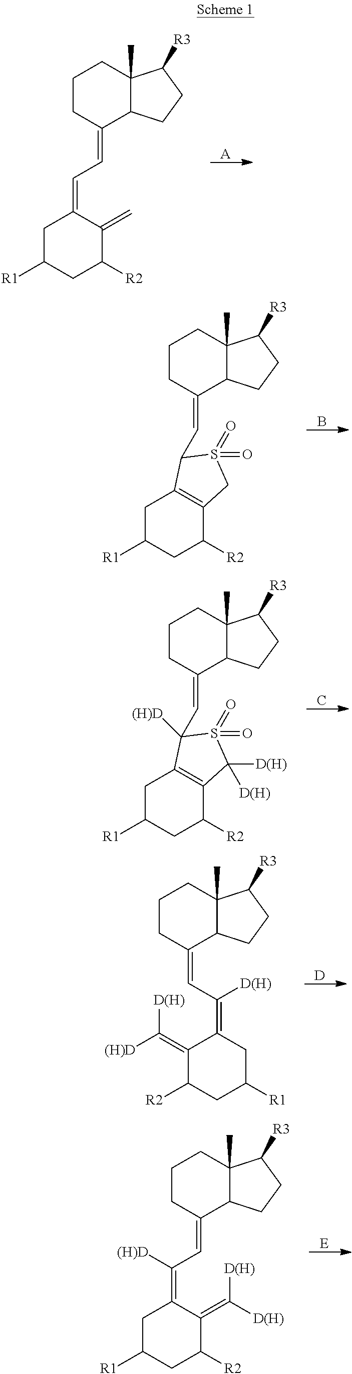

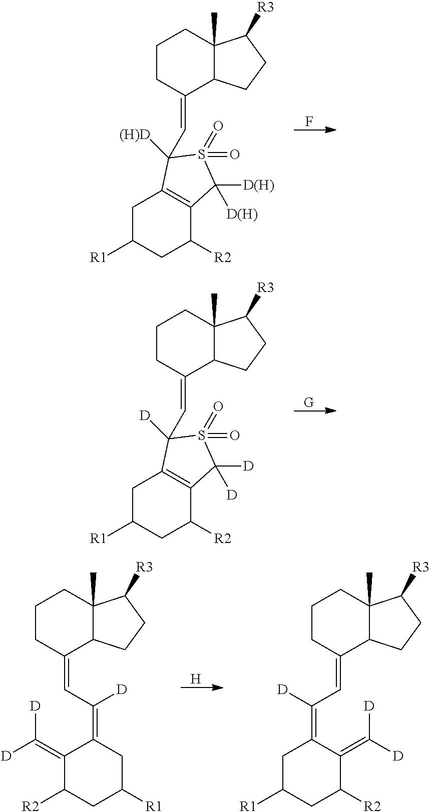

The process is comprised of the following steps:

### Step A

The preparation of the sulfur dioxide adduct is readily available by condensation of sulfur dioxide onto the vitamin or vitamin analog as described by Reischl and Zbiral in Helvetica Chimica Acta 62(6) 1763 (1979). It is preferred that the reaction be carried out without solvent. The reaction is stirred until such time as the reaction is complete, typically 15 to 60 minutes, as judged by thin layer chromatography. The sulfur dioxide is removed and the product obtained directly carried on.

### Step B

The adduct with sulfur dioxide is dissolved in a solvent having a labile deuterium atom, typically methanol-D or ethanol-D, and is treated under basic conditions to effect the exchange of hydrogen with deuterium. The base may be sodium hydroxide, sodium methoxide or sodium ethoxide. Those skilled in the art will recognize other possible bases to effect the desired labeling reaction.

### Step C

The deuterated sulfur dioxide adduct is heated until all of the sulfur dioxide has been extruded from the molecule as judged by thin layer chromatography. This process typically takes 2 hours.

### Step D

The partially deuterated product obtained in Step C is photoisomerized with UV light and an appropriate sensitizer, such as Eosin Y, until the correct double bond geometry at C7 has been obtained as judged by thin layer chromatography.

### Alternative to Step D

Step D may be omitted and the product obtained from Step C directly subjected to the conditions described in Steps E through H.

### Steps E-H

Steps A to D are repeated in their entirety. Flash chromatographic purification gives the final deuterated compound(s) having a minimum amu enhancement of +2.97 and having <0.1% unlabeled material contaminating the final product.

### EXAMPLE 1

### Addition of Sulfur Dioxide to Vitamin D3

2 g of Vitamin D3 was placed in a 500 mL round bottom flask which was equipped with a dry ice condenser. Sulfur dioxide was condensed into the flask (30-40 mL). The reaction was initially bright yellow and slowly faded. The dry ice was removed and the sulfur dioxide was allowed to evaporate. When the solution became thick near the end of the sulfur dioxide evaporation a vacuum was applied to remove the last traces of sulfur dioxide giving a white glassy product. This was used directly in the next reaction.

### EXAMPLE 2

### Addition of Sulfur Dioxide to Vitamin D2

500 mg of Vitamin D2 was placed in a 100 mL round bottom flask equipped with a dry ice condenser and was cooled to −40° C. Sulfur dioxide was condensed into the flask (10-15 mL). The cooling bath was removed and the yellow solution was stirred for an hour. The dry ice was removed and the sulfur dioxide was allowed to evaporate. When the solution became thick near the end of the sulfur dioxide evaporation a vacuum was applied to remove the last traces of sulfur dioxide giving a white glassy product. This was used directly in the next reaction.

### EXAMPLE 3

### Addition of Sulfur Dioxide to 25-Hydroxyvitamin D3

90 mg of 25-Hydroxyvitamin D3 was placed in a 50 mL round bottom flask equipped with a dry ice condenser and was cooled to −40° C. Sulfur dioxide was condensed into the flask (10-15 mL). The cooling bath was removed and the yellow solution was stirred for an hour. The dry ice was removed and the sulfur dioxide was allowed to evaporate. When the solution became thick near the end of the sulfur dioxide evaporation a vacuum was applied to remove the last traces of sulfur dioxide giving a white glassy product. This was used directly in the next reaction.

### EXAMPLE 4

### Addition of Sulfur Dioxide to 25-Hydroxyvitamin D2

The sulfur dioxide adduct of 25-Hydroxyvitamin D2 was obtained in exactly the same manner as 25-Hydroxyvitamin D3 as described in Example 3.

### EXAMPLE 5

Deuteration and Extrusion of Sulfur Dioxide from Vitamin D3

The vitamin D3 sulfur dioxide adduct was dissolved in methanol-D (99.5% D). To this was added 1.4 mL of D₂O. Potassium t-butoxide (8.3 g) was added and the pale yellow solution stirred at room temperature. The reaction was then heated to reflux to eliminate the sulfur dioxide. Reaction was monitored by TLC (4:1 benzene/EtOAc) and took approximately 2 hours to go to completion. The reaction was cooled to room temp and concentrated. The residue was treated with D₂O and the white precipitate collected by filtration, washed with D₂O and then dried.

### EXAMPLE 6

Deuteration and Extrusion of Sulfur Dioxide from Vitamin D2

Deuteration and extrusion of sulfur dioxide from the Vitamin D2 sulfur dioxide adduct was achieved in exactly the same manner as described in Example 5.

### EXAMPLE 7

Deuteration and Extrusion of Sulfur Dioxide from 25-Hydroxyvitamin D3

The 25-Hydroxyvitamin D3 sulfur dioxide adduct was dissolved in 20 mL of methanol-D (99.5%) and treated with 288 mg of potassium t-butoxide and 0.5 mL of D₂O. The reaction was stirred at room temperature and then heated at reflux for 2 hours to eliminate the sulfur dioxide. The reaction was cooled to room temp and concentrated. The residue was treated with D₂O and extracted with methylene chloride. The organic extract was dried and concentrated to give the product.

### EXAMPLE 8

Deuteration and Extrusion of Sulfur Dioxide from 25-Hydroxyvitamin D2

Deuteration and extrusion of sulfur dioxide from the 25-Hydroxyvitamin D2 sulfur dioxide adduct was achieved in exactly the same manner as described in Example 7.

### EXAMPLE 9

### Photoisomerization of the Partially Deuterated Vitamin D3 Analog

The partially deuterated vitamin D3 analog from example 5 was dissolved in ethanol and a small amount of Eosin Y added. The solution was exposed to a UV lamp until isomerization was complete as judged by thin layer chromatography (9:1 benzene/EtOAc). Purification was by flash chromatography (9:1 benzene/EtOAc).

### EXAMPLE 10

### Photoisomerization of the Partially Deuterated Vitamin D2 Analog

The partially deuterated vitamin D2 analog from example 6 was photoisomerized as described in example 9.

### EXAMPLE 11

### Photoisomerization of the Partially Deuterated 25-Hydroxyvitamin D3 Analog

The product from example 7 was dissolved in ethanol and a spatula tip of Eosin Y added. The solution was exposed to a UV lamp until isomerization was complete as judged by thin layer chromatography (4:1 benzene/EtOAc). Purification was carried out by flash chromatography (4:1 benzene/EtOAc).

### EXAMPLE 12

### Re-Deuteration of Partially Deuterated Vitamin D3

The partially deuterated vitamin D3 obtained from either example 5 or example 9 was re-subjected to the entire sequence of chemistry described above in examples 1, 5 and 9. Following this procedure, deuterated vitamin D3 was obtained having an isotopic enrichment of ≧2.97 D/molecule. The product had <0.1% unlabelled vitamin D3 remaining making the end product of this reaction sequence suitable for use as a standard for mass spectroscopy.

### EXAMPLE 13

### Re-Deuteration of Partially Deuterated Vitamin D2

The partially deuterated vitamin D2 obtained from either example 6 or example 10 was re-subjected to the entire sequence of the mistry described above in examples 2, 6 and 10. Following this procedure, deuterated vitamin D2 was obtained having an isotopic enrichment of ≧2.97 D/molecule. The product had <0.1% unl abelled vitamin D2 remaining making the end product of this reaction sequence suitable for use as a standard for mass spectroscopy.

### EXAMPLE 14

### Re-Deuteration of Partially Deuterated 25-Hydroxyvitamin D3

The partially deuterated 25-Hydroxyvitamin D3 obtained from either example 7 or example 11 was re-subjected to the entire sequence of chemistry described above in examples 3, 7 and 11. Following this procedure, deuterated 25-Hydroxyvitamin D3 was obtained having an isotopic enrichment of ≧2.97 D/molecule. The product had <0.1% unlabeled 25-Hydroxyvitamin D3 remaining making the end product of this reaction sequence suitable for use as a standard for mass spectroscopy.

### EXAMPLE 15

### Re-Deuteration of Partially Deuterated 25-Hydroxyvitamin D2

The partially deuterated 25-Hydroxyvitamin D2 obtained from example 8 was re-subjected to the entire sequence of chemistry described above in examples 4, 8 and 12. Following this procedure, deuterated 25-Hydroxyvitamin D2 was obtained having an isotopic enrichment of ≧2.97 D/molecule. The product had <0.1% unlabelled 25-Hydroxyvitamin D2 remaining making the end product of this reaction sequence suitable for use as a standard for mass spectroscopy.

Among other features of the present invention, it will be appreciated that the compounds produced in accordance therewith can be dissolved in various solutions, including without limitation in methanol, isopropanol, and other organic solvents and, in particular, ethanol is preferred as a solvent as it has been found that such solutions of the compounds in ethanol are capable of providing improved stability for the compounds of the invention, by comparison with other solvents.

Additional embodiments, as well as features, benefits and advantages, of the present invention will be apparent to those skilled in the art, taking into account the foregoing description of preferred embodiments of the invention. It is therefore to be appreciated that the present invention is not to be construed as being in any way limited by the foregoing description of such preferred embodiments, but that various changes and modifications can be made to the invention as specifically described herein, and that all such changes and modifications are intended to be within the scope of the present invention. Any such limitations are only to be construed as being defined by the claims appended hereto.

What is claimed is:

1. A process for preparing deuterated vitamin D compounds having a characteristic 5Z, 7E, 10(19) triene system comprising the steps of:
(a) reacting compound 1 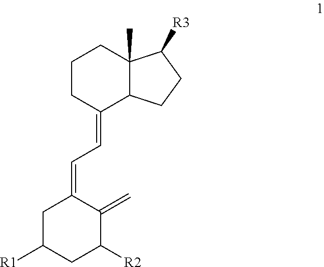
In the presence of sulfur dioxide to produce compound 2: 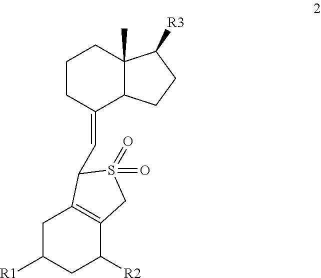
(b) reacting compound 2 in basic conditions in a solvent with labile deuterium to produce compound 3: 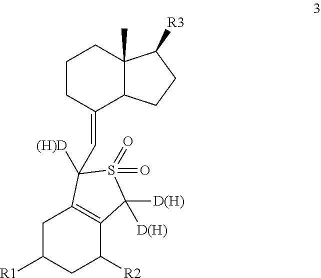
(c) heating compound 3 to produce compound 4: 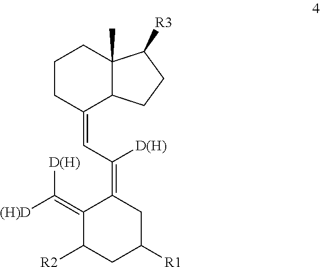
(d) reacting compound 4 in the presence of sulfur dioxide to produce compound 5: 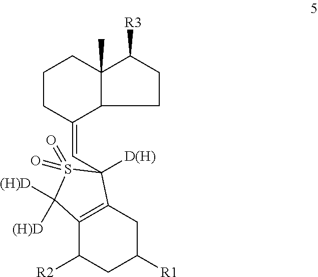
(e) reacting compound 5 in basic conditions in a solvent with labile deuterium to produce compound 6: 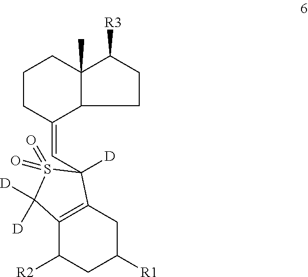
(f) heating compound 6 to produce compound 7: 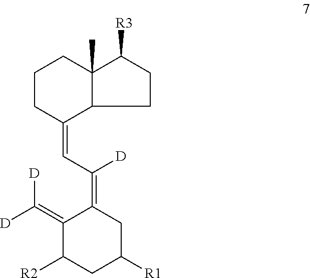
(g) photoisomerizing compound 7 with UV light to produce compound 8: 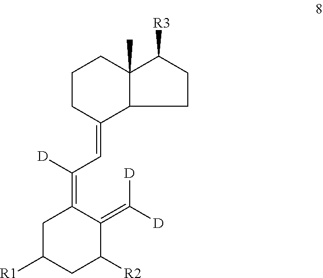
Wherein where R1=H or OH, R2=H or OH and R3 is either 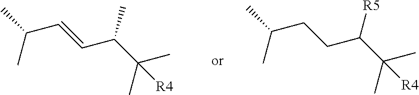
where R4 and R5 are H or OH.

2. A process for preparing deuterated vitamin D compounds having a characteristic 5Z, 7E, 10(19) triene system comprising the steps of:
(a) reacting compound 1: 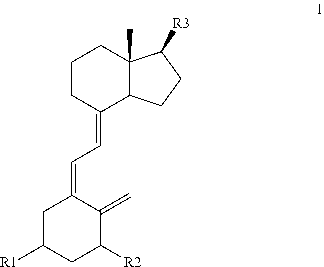
In the presence of sulfur dioxide to produce compound 2: 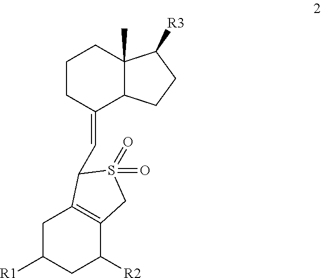
(b) reacting compound 2 in basic conditions in a solvent with labile deuterium to produce compound 3: 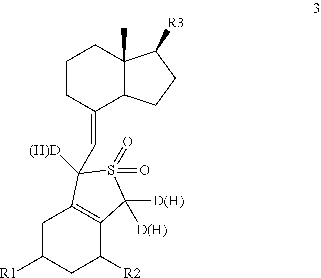
(c) heating compound 3 to produce compound 4: 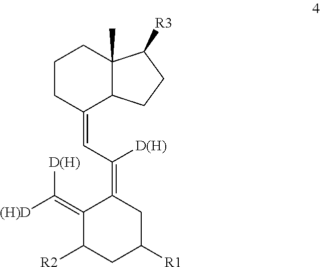
(d) photoisomerizing compound 4 with UV light to produce compound 5: 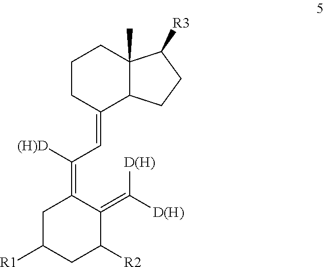
(e) reacting compound 5 with sulfur dioxide to produce compound 6: 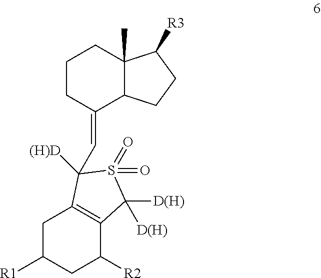
(f) reacting compound 6 in basic conditions in a solvent with labile deuterium to produce compound 7: 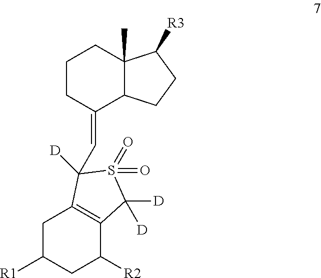
(g) heating compound 7 to produce compound 8: 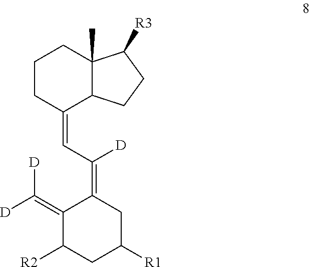
(h) photoisomerizing compound 8 with UV light to produce compound 9: 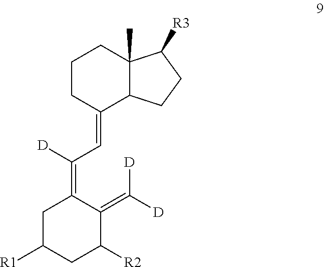
Wherein where R1=H or OH, R2=H or OH and R3 is either 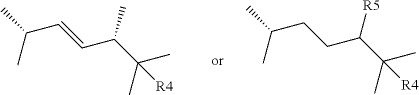
where R4 and R5 is H or OH.

3. A compound prepared according to the process claimed in claim 1 or claim 2, said product being suitable for use as a standard for mass spectroscopy.

4. A compound having the formula: 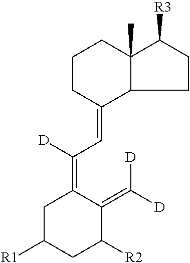
Wherein where R1=H or OH, R2=H or OH and R3 is either 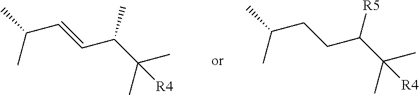
where R4 and R5 are H or OH.

5. A compound having the formula: 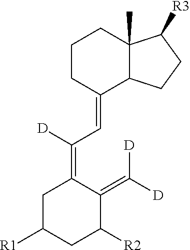
Wherein where R1=H or OH, R2=H or OH and R3 is either 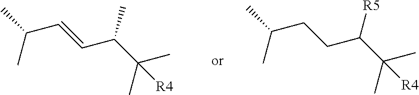
where R4 and R5 is H or OH.

6. The compound of claim 4, wherein in addition said compound is in a solution.

7. The compound of claim 5, wherein in addition said compound is in a solution.

8. The compound of claim 4, wherein said compound is in a solution of an organic solvent selected from the group consisting of methanol, ethanol and isopropanol.

9. The compound of claim 5, wherein said compound is in a solution of an organic solvent selected from the group consisting of methanol, ethanol and isopropanol.

10. The compound of claim 3, wherein said compound is in a solution of an organic solvent selected from the group consisting of methanol, ethanol and isopropanol.
